(19) United States
(12) Patent Application Publication
(10) Pub. No.: US 2026/0182892 A1
(43) Pub. Date: Jul. 2, 2026

(54) Restraining Device with EKG Sensor and Wireless Connectivity

(71) Applicant: 4Sight Labs Inc., San Francisco, CA (US)

(72) Inventor: Ian Andrew Osteyee, Keene, NY (US)

(73) Assignee: 4Sight Labs Inc., San Francisco, CA (US)

(21) Appl. No.: 19/130,152

(22) PCT Filed: Nov. 15, 2023
(86) PCT No.: PCT/US2023/079838

§ 371 Date: May 15, 2025

Related U.S. Application Data
(60) Provisional application No. 63/425,762, filed on Nov. 16, 2022.

(51) Int. Cl. A61B 5/332; A61B 5/00; A61B 5/256; A61B 5/282; E05B 75/00

(52) U.S. Cl. CPC A61B 5/332; A61B 5/0006; A61B 5/256; A61B 5/282; A61B 5/6824; A61B 5/746; E05B 75/00; A61B 2503/12

(57) ABSTRACT

A restraining device, and method thereof, for shackling first and second wrists of an individual with lockable first and second cuffs extending from opposite ends of a link that electrically isolates the first and second cuffs, where the restraining device includes an electrocardiogram (EKG) sensor having a first input electrically coupled to an electrically conductive portion of the first cuff, such as a first double strand adapted to contact the first wrist, and a second input electrically coupled to an electrically conductive portion of the second cuff, such as a second double strand adapted to contact the second wrist. The restraining device further includes a wireless communications module configured to send EKG data and/or alert messages to an electronic device.

### FIELD

This disclosure relates to restraining devices for law enforcement, and more particularly, to handcuffs that include electronics for monitoring physiological signals of a person wearing the handcuffs and for wirelessly transmitting physiological information and/or alert messages to a receiving device. Even more particularly, this disclosure relates to handcuffs comprising a sensor, for monitoring electrical heart activity, and a wireless communications module, for sending sensor data or related information to an electronic device.

### BACKGROUND

Law enforcement activity occasionally requires a law enforcement officer to restrain a person using a restraining device such as handcuffs. The restrained person, who may be referred to herein as a detainee, may have preexisting physical or mental conditions, or the detainee may have been physically injured in connection with being detained and/or restrained, or the detainee may experience anxiety, distress, agitation, or other emotions as a result of being detained and/or restrained. Such physical, mental, and/or emotional states may result in divergent physiological conditions of the detainee, such as elevated or irregular heart rate or elevated blood pressure, that may cause additional injury or even death of the detainee. It is therefore beneficial to be able to monitor certain physiological conditions of the detainee and to receive notifications when such physiological conditions exceed certain thresholds. Because the detainee may be uncooperative or unresponsive, it is especially beneficial to be able to monitor certain physiological conditions of the detainee without requiring the use of dedicated monitoring equipment or the cooperation of the detainee.

### SUMMARY

The disclosed embodiments provide for a restraining device that includes a sensor for monitoring certain physiological conditions of a person restrained by the restraining device (a detainee). In a preferred embodiment, the restraining device is a pair of handcuffs, but other restraining devices and combinations thereof are within the scope of the invention, such as flexible restrains, disposable restraints, belly chains, leg irons, and so on. Hereafter the term “handcuffs” includes any such restraining device. In a preferred embodiment, the sensor is an electrocardiogram (EKG; also abbreviated ECG) heart-rate sensor that detects electrical activity of the heart. However, other heart-rate sensors, and sensors that detect other physiological conditions, are within the scope of the invention, such as a photoplethysmography (optical) sensor that detects changes in volume of a blood vessel. The terms “sensor” and “monitor” may be used interchangeably herein.

The disclosed embodiments further provide for a restraining device that includes a wireless transmitter for sending, to one or more electronic devices, physiological data of the detainee and/or for sending alert messages when the monitored physiological conditions exceed certain thresholds. Examples of an electronic device include a mobile phone, a tablet, a laptop computer, and so on. An electronic device may receive transmissions directly from the restraining device, for example via Bluetooth wireless protocol. Alternatively or additionally, an electronic device may receive physiological data and/or alert messages from the restraining device via one or more communications networks, for example a cellular network, where the restraining device transmits the physiological data and/or alert messages to a gateway device, for example a cellular tower.

The disclosed embodiments further provide for presenting alerts to an individual, such as a nearby law enforcement officer, when the monitored physiological conditions of the detainee exceed certain thresholds or when certain physiological patterns are detected. The alerts may take any suitable form, such as stimulus produced by an electronic device that is visual (e.g., graphics and/or text on a display screen or flashing lights), audible (e.g., bells or sirens), or tactile (e.g., vibrations of a wristwatch/smartwatch). The terms “notification” and “alert” may be used interchangeably herein.

In one embodiment, the restraining device is adapted for shackling first and second wrists of a detainee, where the restraining device comprises: lockable first and second cuffs extending from opposite ends of a link and electrically isolated thereby; and an electronics module comprising an EKG sensor having a first input electrically coupled to a first electrode and a second input electrically coupled to a second electrode; where an electrically conductive portion of the first cuff, adapted to contact the first wrist, comprises the first electrode and an electrically conductive portion of the second cuff, adapted to contact the second wrist, comprises the second electrode.

In one embodiment, the electrically conductive portion of the first cuff comprises a first double strand; and the electrically conductive portion of the second cuff comprises a second double strand.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings.

FIG. 1 shows a conventional pair of handcuffs.

FIG. 2A shows plan view of an embodiment of the restraining device where a pair of cuffs is joined via a rigid link housing an electronics module at a medial location thereof, and FIG. 2B shows an elevation view thereof.

FIG. 3 shows a cross-section of the embodiment of FIG. 2A taken along line A-A in FIG. 2B.

FIG. 4 shows an exemplary link that joins the cuffs of the embodiment shown in FIG. 2.

FIG. 5 shows several components of an electronics module of an embodiment of the restraining device.

FIG. 6 shows an exemplary electrical interconnection between an EKG sensor and first and second cuffs of an embodiment of the restraining device.

FIGS. 7A-7C show an electrical path utilized by an EKG sensor of an embodiment of the restraining device.

FIGS. 8A-8C show various communications channels between a restraining device and an electronic device.

FIG. 9 shows an embodiment of the restraining device where a pair of cuffs is joined by a link comprising one or more hinges and housing an electronics module at a medial location thereof.

FIG. 10 shows an embodiment of the restraining device where a pair of cuffs is joined by a link comprising a hinge and housing an electronics module adjacent to one of the cuffs.

FIG. 11A shows an embodiment of the restraining device where a pair of cuffs is joined by a link comprising one or more flexible members and housing an electronics module adjacent to one of the cuffs, and FIG. 11B shows an exemplary series connection of the one or more flexible members.

FIG. 12 shows an embodiment of the restraining device where a pair of cuffs is joined by a link comprising one or more hinges and housing an electronics module at a medial location thereof, where the restraining device is configured to fold in half for compact storage.

FIG. 13 shows an embodiment of the restraining device where a pair of cuffs is joined by a link comprising a flexible webbing and housing an electronics module adjacent to one of the cuffs.

### DETAILED DESCRIPTION

The present disclosure may be more readily understood by reference to the following detailed description and the accompanying drawings, which form a part of this disclosure. This disclosure is not limited to the specific devices, methods, conditions, or parameters described and/or shown herein, and the terminology used herein is for the purpose of describing particular embodiments by way of example only and is not intended to be limiting of disclosed embodiments or inventions. For example, “left,” “right,” “clockwise,” and “counterclockwise” may be used as specific examples of generally opposite lateral or rotational directions, respectively. Also, as used in the specification and including the appended claims, the singular forms “a,” “an,” and “the” include the plural, and reference to a particular numerical value includes at least that particular value, unless the context clearly dictates otherwise.

The following numerals are used to describe various features of the embodiments.
200 restraining device
210 first cuff
220 second cuff
230 double strand
240 single strand
250 strand rivet
260 cheek plate
270 keyway
280 double lock
290 ratchet teeth
300 ratchet pawl
310 link
320 fasteners
330 through-hole
340 separation
350 housing
360 electronics module
370 battery
380 power controller
390 wireless transceiver
400 antenna
410 processor
420 EKG sensor
430a,b electrode I/O
440a,b electrical interconnect
450 electrical path
460 wireless channel
470 receiving device
480 gateway device
500 hinge
510 flexible member

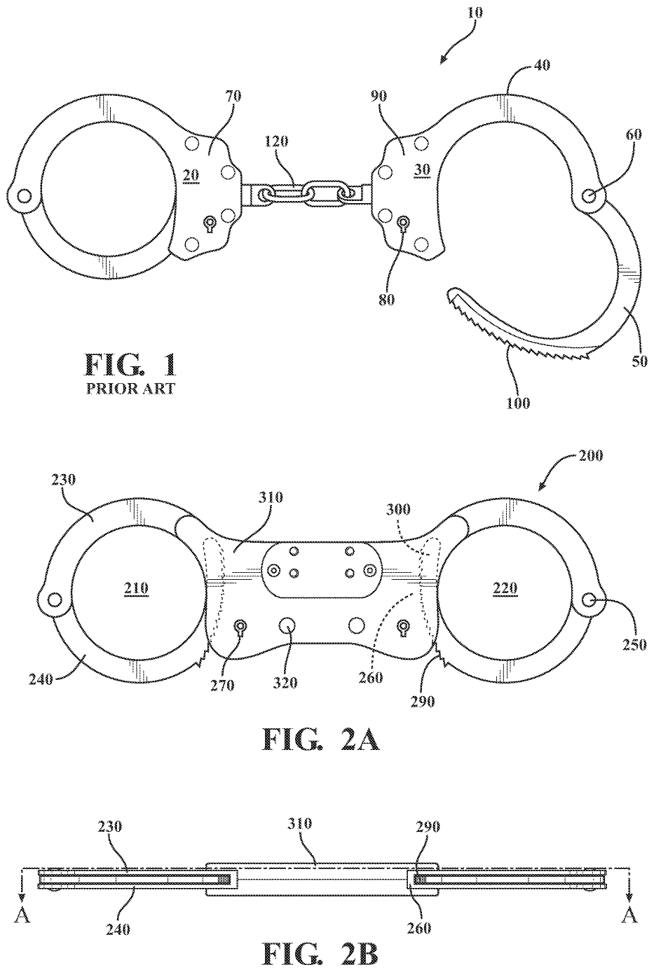

FIG. 1 shows an exemplary pair of handcuffs 10 comprising a first cuff 20 and a second cuff 30 each configured to encircle respective wrists of a detainee. Each cuff comprises a double strand 40 extending from a cheek plate 70 and a single strand 50 pivotally attached to the double strand 40 via strand rivet 60. The double strand 40 is adapted to receive the single strand 50 therethrough when the single strand 50 pivots about the strand rivet 60. Each single strand 50 includes a plurality of ratchet teeth 100 on a distal end thereof, which are adapted to interface with a respective ratchet pawl 110 disposed within each cheek plate 70. A keyway 80 is provided within each cheek plate 70 to permit a key to engage with and lock the ratchet pawl 110, thereby preventing the respective single strand 50 from pivoting open or pivoting further closed. The cheek plates 70 are connected via a link 120, which may be any suitable component that may be rigid, flexible, hinged, articulated, and so on.

FIGS. 2A-2B show an embodiment of a restraining device 200 disclosed herein, comprising a first cuff 210 and a second cuff 220 each comprising: a double strand 230 extending from a cheek plate 260, a single strand 240 pivotally attached to the double strand 230 via a strand rivet 250, ratchet teeth 290 disposed on a distal end of the single strand 240 and adapted to interface with a ratchet pawl 300 disposed within the cheek plate 260, and a keyway 270 provided within the cheek plate 260 (and through a portion of the link 310) to permit a key to engage with and lock the ratchet pawl 300. The link 310 is shown as a rigid member; however, in some embodiments the link may be rigid, flexible, hinged, articulated, and so on. For example, in some embodiments the link 310 may comprise a flexible strap, a webbing, a cable, a hinge, and so on.

The first cuff 210 and the second cuff 220 each comprise a material having sufficient mechanical strength for use in handcuffs, and each comprise a material that is electrically conductive, such as certain metals (e.g., steel). Specifically, the cheek plate 260 and the double strand 230 of a respective cuff 210 or 220 may be formed of a continuous piece of electrically conductive metal or from multiple pieces of electrically conductive metal that are integrally joined via welds, rivets, fasteners, or any other suitable means. The single strand 240 may also be formed of an electrically conductive material, which may electrically couple to the double strand 230 via the strand rivet 250 and which may electrically couple to the cheek plate 260 via the ratchet teeth 290 contacting the ratchet pawl 300 when the respective cuff 210 or 220 is in a closed position.

The link 310 comprises a material having sufficient mechanical strength for use in handcuffs, and is comprised of a material that is electrically insulative, such as certain plastics (e.g., polyamide, acrylonitrile butadiene styrene, polyetheretherketone, polyamideimide, and so on). The link 310 may be produced by any suitable means, for example, machining or injection molding, and the link 310 may be one piece or multiple pieces secured together. The first cuff 210 and the second cuff 220 may be secured to the link 310 via any suitable means, such as fasteners 320 as shown in FIG. 2A, or via glue, plastic-injection over-molding, welding, compression fittings, and so on. Each fastener 320 may be any suitable type, such as a screw, a bolt coupled to a nut, a post coupled to a nut, a one-or two-piece rivet, a spring pin, and so on.

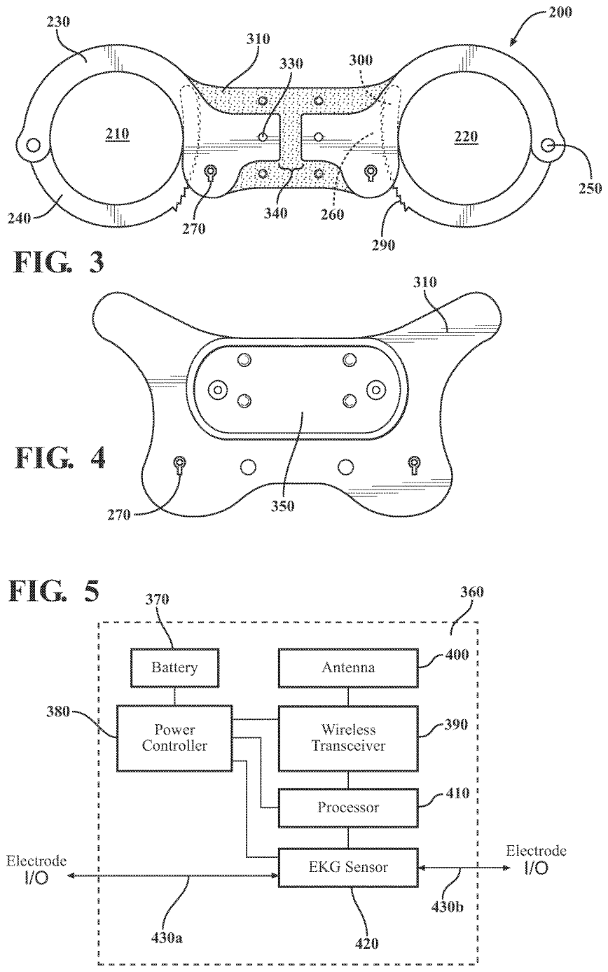

FIG. 3 shows a cross-section of the restraining device 200 shown in FIG. 2B taken along line A-A in FIG. 2B (or alternatively, showing the restraining device 200 wherein the link 310 is a multipart assembly and one part has been removed). In this embodiment, the cheek plate 260 of each cuff 210 and 220 includes a through-hole 330 in which a respective fastener 320 (not shown in FIG. 3) may be placed. The link 310 provides a separation 340 between the first cuff 210 and the second cuff 220. The separation may comprise an air gap or it may comprise a dielectric (i.e., an insulator or insulative material), thereby electrically isolating the first cuff 210 from the second cuff 220. Although the separation 340 in FIG. 3 is shown as a gap in the plane of the cross-section along a longitudinal axis of the link 310, the separation 340 may be oriented or arranged in any suitable manner, for example along a transverse axis of the link 310 in or perpendicular to the plane of the cross-section.

FIG. 4 shows the link 310, which provides a housing 350 for storing and protecting an electronics module 360 of the restraining device 200. The housing 350 may comprise a recess in the link 310 and a cover secured thereover, or the housing 350 may comprise a self-contained unit that may be permanently or detachable secured to the link 310. The electronics module 360 may be permanently or detachably secured within the housing 350.

FIG. 5 shows a block diagram of an exemplary electronics module 360. An electrocardiogram (EKG or ECG) sensor 420 sends and/or receives electrical signals from at least two electrode input/outputs (I/Os) 430a and 430b. An electrical signal may be a voltage signal or a current signal. A processor 410 may process or otherwise prepare the EKG data captured by the EKG sensor 420 from one or both of its electrode I/Os 430a and 430b for transmission via a wireless transceiver 390 coupled to an antenna 400. The processor 410 may be any suitable electronic component capable of executing instructions, such as a microprocessor, a microcontroller, an application-specific integrated circuit (ASIC), a field-programmable gate array (FPGA), and so on. The electronic components may be powered via any suitable energy storage element, such as a battery 370, and such energy may be distributed or otherwise controlled via a power controller 380. The processor 410 may control or otherwise coordinate operations of the power controller 380, the wireless transceiver 390, and/or the EKG sensor 420. The battery 370 may be single-use or rechargeable, and may be integral or replaceable. A rechargeable battery 370 may be recharged by any suitable means, such as non-contact inductive charging.

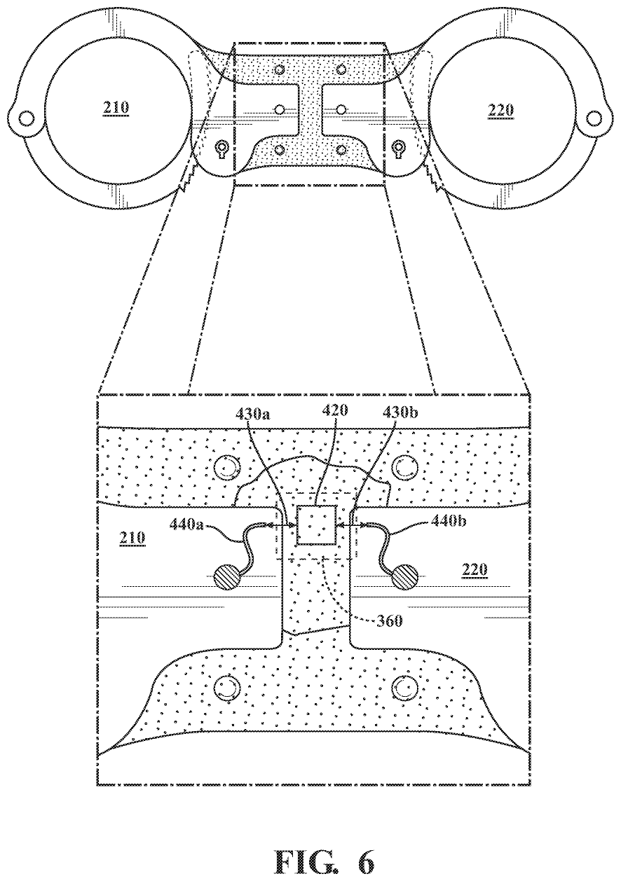

FIG. 6 shows an exemplary electrode interconnection of the EKG sensor 420 and the cuffs 210 and 220 of the restraining device 200. A first electrode I/O 430a of the EKG sensor 420 is electrically coupled to an electrically conductive portion of the first cuff 210, for example a steel cheek plate 260 and/or a steel double strand 230 thereof, via a first electrical interconnect 440a that is disposed at least partly within the link 310. Such electrical coupling enables the entire electrically conductive portion of the first cuff to serve as a first electrode for the EKG sensor 420. Similarly, a second electrode I/O 430b is electrically coupled to an electrically conductive portion of the second cuff 220, for example a steel cheek plate 260 and/or a steel double strand 230 thereof, via a second electrical interconnect 440b that is disposed at least partly within the link 310. Such electrical coupling enables the entire electrically conductive portion of the second cuff to serve as a second electrode for the EKG sensor 420. The electrical interconnects 440a and 440b may be any suitable element capable of electrically coupling a respective cuff 210 or 220 to a respective electrode I/O 430a or 430b, such as an electrical wire, a conductive fastener (e.g., a bolt), a spring clip or spring contact, a pogo pin, a dowel, a weld or solder contact, and so on. The electrode I/Os 430a and 430b may comprise a microchip lead (e.g., a pin or a ball); a printed circuit board (PCB) trace, pad, or via (a metallized through-hole); a spring clip or spring contact; and so on.

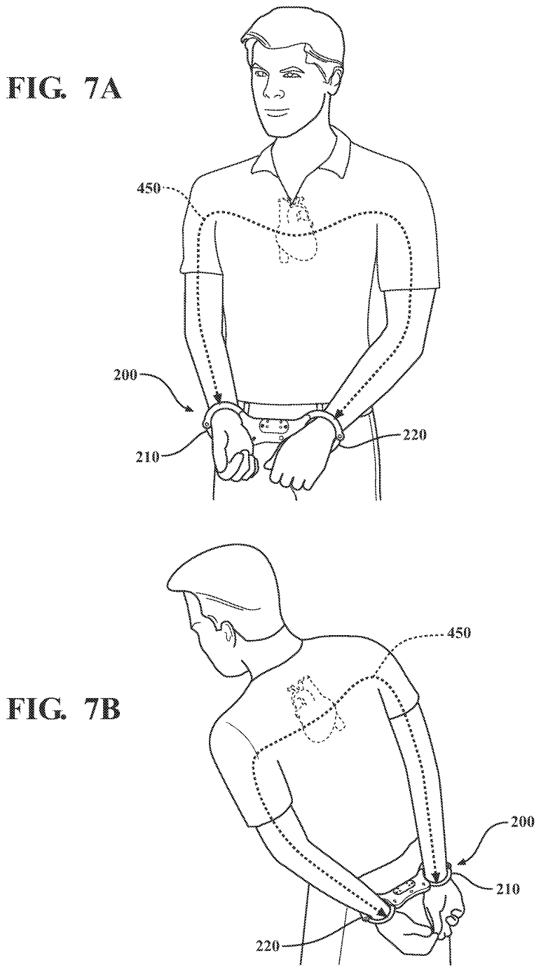

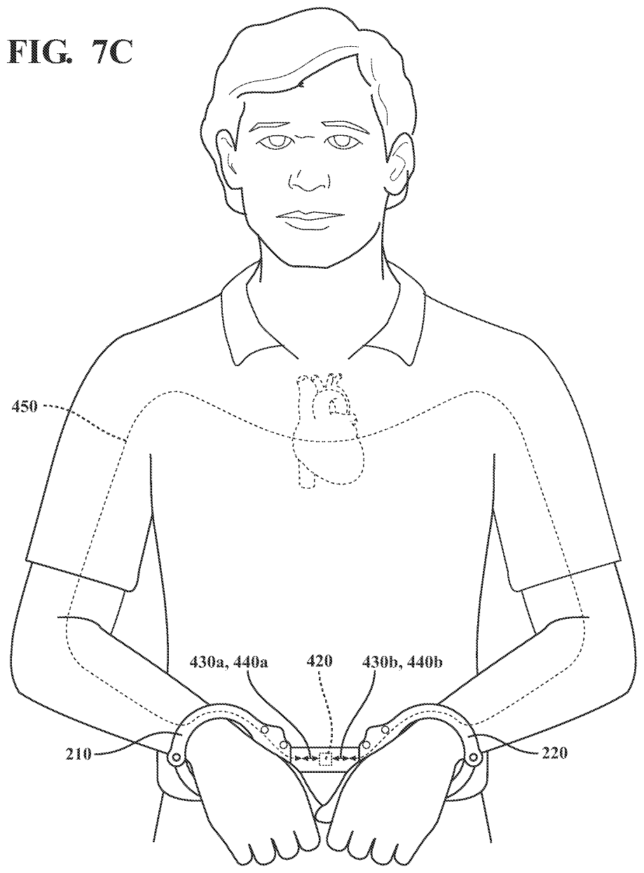

How an EKG sensor detects electrical activity (electrical potentials) of a heart is known in the art and is not detailed herein, other than to note that for an EKG sensor to effectively detect electrical activity of a heart, there should be an electrical path between the electrodes of the EKG sensor that traverses the heart. FIGS. 7A-7C show a detainee wearing the restraining device 200, where there is an electrical path 450 between a first electrode I/O 430a (and its electrode) and a second electrode I/O 430b (and its electrode) that traverses the detainee's heart. Specifically, the electrical path 450 may comprise: an EKG sensor 420, a first electrode I/O 430a, a first electrical interconnect 440a, a conductive portion of a first cuff 210, a physical contact between the skin of a first wrist and the conductive portion of the first cuff 210, a first arm, the chest, a second arm, a physical contact between the skin of a second wrist and a conductive portion of a second cuff 220, the conductive portion of the second cuff 220, a second electrical interconnect 440b, a second electrode I/O 430b, and finally back to the EKG sensor 420.

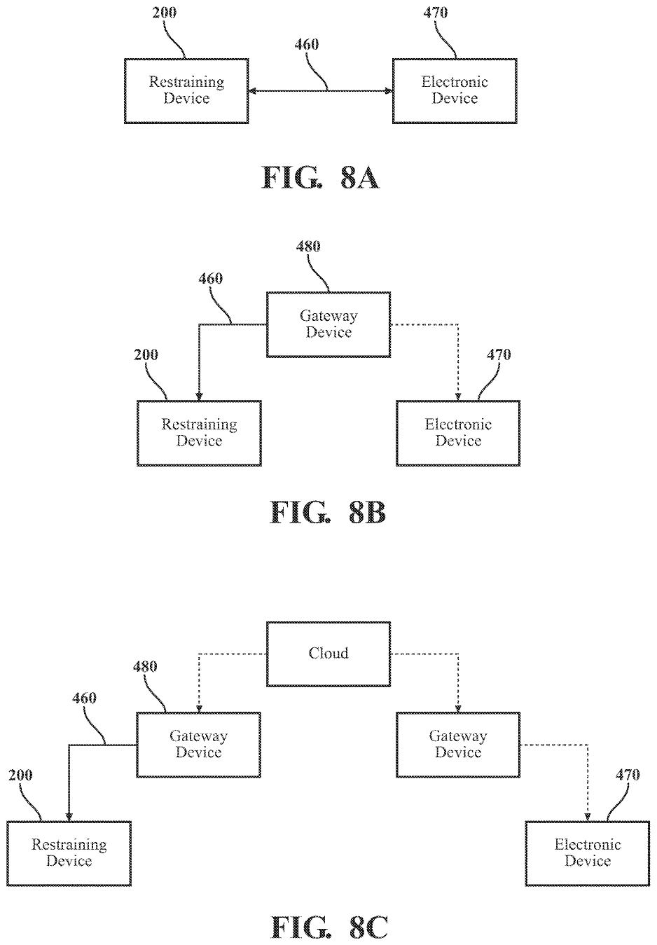

FIGS. 8A-8C show several ways in which the EKG data collected by the EKG sensor 420, or alert messages based on such data, may be wirelessly transmitted to an electronic device, such as a mobile phone, tablet, laptop computer, connected vehicle, smartwatch, a body-worn multimedia device (such as augmented reality glasses and/or an audio headset), and so on. FIG. 8A depicts a direct wireless transmission from the restraining device 200 to the electronic device 470 via a wireless channel 460. Such direct wireless transmission may be achieved by any suitable protocol, for example Bluetooth, Bluetooth Low Energy, ZigBee, Z-Wave, 6LoWPAN, Thread, ANT & ANT+, DigiMesh, EnOcean, Dash7, WirelessHART, NFC, RFID, and so on. FIGS. 8B and 8C depict the restraining device 200 wirelessly transmitting to a gateway device 470, which may be achieved by any suitable protocol, for example any of the protocols listed above, as well as WiFi-ah (HaLow), 2G (GSM), 3G, 4G, LTE (Cat 0, 1, & 3), LTE-M1, NB-IoT, 5G, SigFox, LoRaWAN, Ingenu, Weightless-N, Weightless-P, Weightless-W, and so on. In FIG. 8B, the gateway device 470 relays the transmission to the electronic device 470. In FIG. 8C, the gateway device 470 forwards the transmission to the electronic device via one or more communications networks, for example over the Internet or a cloud network via the TCP/IP protocol. The wireless channel 460 may be unidirectional or bidirectional.

In some embodiments, an absolute or relative location of the restraining device 200 may be determined and utilized for selecting which of a plurality of electronic devices 470 may receive EKG data and/or alert messages, and further, which of a plurality of electronic devices 470 may present EKG data and/or alerts to a respective user thereof. For example, only one or a few electronic devices 470 that are nearest to the restraining device 200 may receive EKG data and/or alert messages or present such EKG data or alerts to a respective user thereof. The restraining device 200 may include any suitable means for determining or assisting in the determining of its location, for example, a GPS module or a module for determining wireless signal-strength. Methods to determine absolute or relative location of a device are known in the art and are therefore not detailed herein.

In some embodiments, the restraining device may receive wireless communications transmissions, for example, to update firmware or software or to upload analytical models for the analysis or processing of EKG data. The processor 410 may utilize such analytical models to determine appropriate thresholds for creating or sending of alert messages.

FIGS. 9-13 show alternate embodiments of the restraining device 200.

FIG. 9 shows an embodiment of the restraining device 200 where a first cuff 210 and a second cuff 220 are each secured to a link 310 via one or more hinges 500, and a housing 350 for an electronics module 360 (not visible) is disposed near a medial portion of the link 310. At least one hinge 500 on each side of the link 310 comprises a conductive element, such as an electrical wire, that electrically couples a conductive portion of a respective cuff 210 and 220 to a respective electrode I/O 430a and 430b.

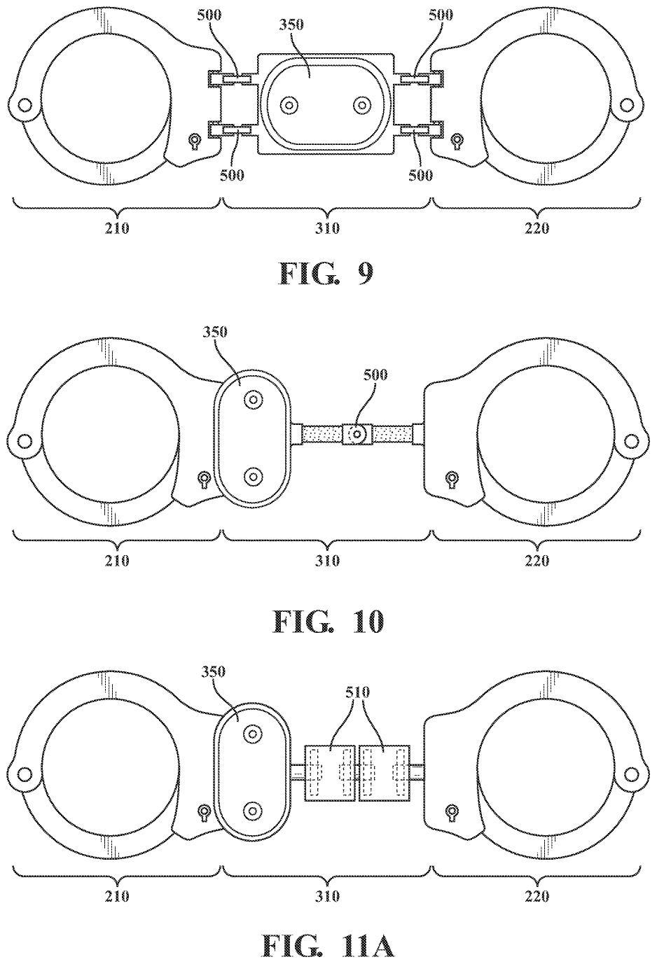

FIG. 10 shows an embodiment of the restraining device 200 where a first cuff 210 is directly secured to a link 310 and a second cuff 220 is secured to the link 310 via one or more hinges 500, and a housing 350 for an electronics module 360 (not visible) is disposed adjacent to the first cuff 210. At least one hinge 500 comprises a conductive element, such as an electrical wire, that electrically couples a conductive portion of the second cuff 220 to a second electrode I/O 430b.

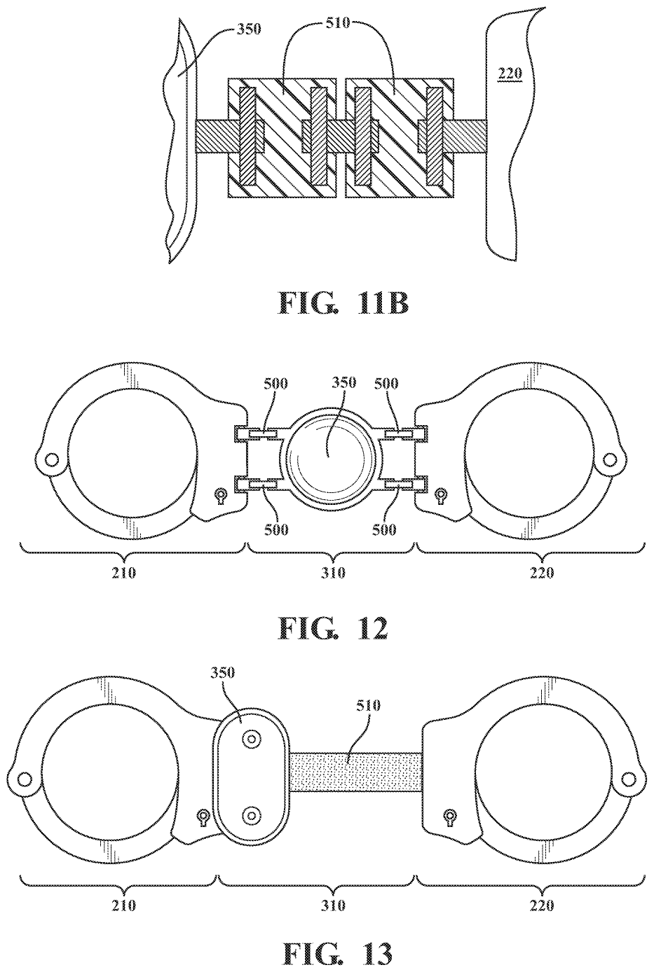

FIG. 11A shows an embodiment of the restraining device 200 where a first cuff 210 is directly secured to a link 310 and a second cuff 220 is secured to the link 310 via one or more flexible members 510, such as rubber or synthetic webbing, and a housing 350 for an electronics module 360 (not visible) is disposed adjacent to the first cuff 210. The one or more flexible members 510 (joined in series in FIG. 11A) comprise one or more conductive elements, such as an electrical wire and/or a metal link, that electrically couples a conductive portion of the second cuff 220 to a second electrode I/O 430b. FIG. 11B shows an exemplary series connection of the one or more flexible members 510 joined via metal screws or posts and a metal link.

FIG. 12 shows an embodiment of the restraining device 200 where a first cuff 210 and a second cuff 220 are each secured to a link 310 via one or more hinges 500, and a housing 350 for an electronics module 360 (not visible) is disposed near a medial portion of the link 310, where the restraining device is configured to be folded in half for compact storage. At least one hinge 500 on each side of the link 310 comprises a conductive element, such as an electrical wire, that electrically couples a conductive portion of a respective cuff 210 or 220 to a respective electrode I/O 430a or 430b.

FIG. 13 shows an embodiment of the restraining device 200 where a first cuff 210 is directly secured to a link 310 and a second cuff 220 is secured to the link 310 via a flexible member 510, such as a Kevlar webbing, and a housing 350 for an electronics module 360 (not visible) is disposed adjacent to the first cuff 210. The flexible member comprises a conductive element, such as an electrical wire, that electrically couples a conductive portion of the second cuff 220 to a second electrode I/O 430b.

Some implementations of the restraining device disclosed herein include a restraining device for shackling first and second wrists of an individual, the restraining device comprising: lockable first and second cuffs extending from opposite ends of a link and electrically isolated thereby; and an electronics module comprising an electrocardiogram (EKG) sensor having a first input electrically coupled to a first electrode and a second input electrically coupled to a second electrode; wherein an electrically conductive portion of the first cuff, adapted to contact the first wrist, comprises the first electrode and an electrically conductive portion of the second cuff, adapted to contact the second wrist, comprises the second electrode.

Some implementations of the restraining device disclosed herein include a method for monitoring electrical heart activity, the method comprising: shackling first and second wrists of an individual with a restraining device comprising: lockable first and second cuffs extending from opposite ends of a link and electrically isolated thereby; and an electronics module comprising an electrocardiogram (EKG) sensor having a first input electrically coupled to a first electrode and a second input electrically coupled to a second electrode; wherein an electrically conductive portion of the first cuff, that contacts the first wrist, comprises the first electrode and an electrically conductive portion of the second cuff, that contacts the second wrist, comprises the second electrode.

In some implementations, the electrically conductive portion of the first cuff comprises a first double strand; and the electrically conductive portion of the second cuff comprises a second double strand.

In some implementations, the electronics module further comprises: a wireless transmitter communicatively coupled to the EKG sensor and configured to send at least one of EKG data and alert messages to an electronic device.

In some implementations, the electronic device comprises at least one of: a mobile phone, a computer, a tablet, a vehicle, a wristwatch, and a body-worn multimedia device.

In some implementations the electronics module further comprises: a wireless receiver configured to receive messages from an electronic device.

In some implementations, the link comprises a rigid member; and the electronics module is housed within the link.

In some implementations the first and second cuffs are each immovably fixed to the link.

In some implementations, at least one of the first cuff and the second cuff is secured to the link via a hinge.

In some implementations, the link comprises a flexible member; and the electronics module is disposed within a housing on or adjacent to the link.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

1. A restraining device for shackling first and second wrists of an individual, the restraining device comprising:
lockable first and second cuffs extending from opposite ends of a link and electrically isolated thereby; and
an electronics module comprising an electrocardiogram (EKG) sensor having a first input electrically coupled to a first electrode and a second input electrically coupled to a second electrode; wherein
an electrically conductive portion of a first double strand of the first cuff, adapted to contact the first wrist, comprises the first electrode and an electrically conductive portion of a second double strand of the second cuff, adapted to contact the second wrist, comprises the second electrode.

2. (canceled)

3. The restraining device of claim 1 wherein the electronics module further comprises:
a wireless transmitter communicatively coupled to the EKG sensor and configured to send at least one of EKG data and alert messages to an electronic device.

4. The restraining device of claim 3 wherein the electronic device comprises at least one of:
a mobile phone, a computer, a tablet, a vehicle, a wristwatch, and a body-worn multimedia device.

5. The restraining device of claim 1 wherein the electronics module further comprises:
a wireless receiver configured to receive messages from an electronic device.

6. The restraining device of claim 3 wherein:
the link comprises a rigid member; and
the electronics module is housed within the link.

7. The restraining device of claim 6 wherein:
the first and second cuffs are each immovably fixed to the link.

8. The restraining device of claim 6 wherein:
at least one of the first cuff and the second cuff is secured to the link via a hinge.

9. The restraining device of claim 3 wherein:
the link comprises a flexible member; and
the electronics module is disposed within a housing on or adjacent to the link.

10. A method for monitoring electrical heart activity, the method comprising:
shackling first and second wrists of an individual with a restraining device comprising:
lockable first and second cuffs extending from opposite ends of a link and electrically isolated thereby; and
an electronics module comprising an electrocardiogram (EKG) sensor having a first input electrically coupled to a first electrode and a second input electrically coupled to a second electrode; wherein
an electrically conductive portion of a first double strand of the first cuff, that contacts the first wrist, comprises the first electrode and an electrically conductive portion of a second double strand of the second cuff, that contacts the second wrist, comprises the second electrode.

11. (canceled)

12. The method of claim 10 wherein the electronics module further comprises:
a wireless transmitter communicatively coupled to the EKG sensor and configured transmit at least one of EKG data and alert messages to an electronic device.

13. The method of claim 12 wherein the electronic device comprises at least one of:
a mobile phone, a computer, a tablet, a vehicle, a wristwatch, and a body-worn multimedia device.

14. The method of claim 10 wherein the electronics module further comprises:
a wireless receiver configured to receive messages from an electronic device.

15. The method of claim 12 wherein:
the link comprises a rigid member; and
the electronics module is housed within the link.

16. The method of claim 15 wherein:
the first and second cuffs are each immovably fixed to the link.

17. The method of claim 15 wherein:
at least one of the first cuff and the second cuff is secured to the link via a hinge.

18. The method of claim 12 wherein:
the link comprises a flexible member; and
the electronics module is disposed within a housing on or adjacent to the link.
